(19) United States
(12) Patent Application Publication
(10) Pub. No.: US 2024/0417318 A1
(43) Pub. Date: Dec. 19, 2024

(54) METHOD FOR SPRAY COATING A SURFACE ATTACHED HYDROGEL

(71) Applicants: Streck LLC, La Vista, NE (US); ALBERT-LUDWIGS-UNIVERSITÄT FREIBURG, Freiburg (DE)

(72) Inventors: Sean Salamifar, Papillion, NE (US); Nicholas Michael George, Omaha, NE (US); David William Conrad, Tampa, FL (US); Jürgen Rühe, Eichstetten (DE); Thomas Brandstetter, Freiburg (DE); Frank Scherag, Freiburg (DE); Nathan Bentley, Freiburg (DE)

(21) Appl. No.: 18/702,932

(22) PCT Filed: Oct. 20, 2022
(86) PCT No.: PCT/US2022/047199

§ 371 Date: Apr. 19, 2024

Related U.S. Application Data
(60) Provisional application No. 63/257,940, filed on Oct. 20, 2021.

(51) Int. Cl. C03C 17/00; A61J 1/05; A61J 1/14; C03C 17/32

(52) U.S. Cl. CPC C03C 17/004; A61J 1/05; A61J 1/1468; C03C 17/32; C03C 2217/70; C03C 2218/112; C03C 2218/31; C03C 2218/32

(57) ABSTRACT

A coated container having reduced nonspecific adsorption of nonspecific targets can have an interior volume surrounded by one or more walls having one or more interior surfaces, and a coating comprising a spray-coated hydrogel layer surface attached to the one or more interior surfaces, the spray-coated hydrogel having surface roughness with surface features with an average features height of less than 20 nm.

### BACKGROUND

The prevention of non-specific protein adsorption has been previously achieved through various means, such as pre-adsorbing of bovine serum albumin, applying fluorinated coatings, or by using PEG brushes. Protein repellency has also been achieved in surface attached and cross-linked hydrogels. By attaching a cross-linked hydrogel to the surface, the confinement of the polymer chains in the swollen network results in an energetically unfavorable situation for proteins to penetrate the layer. This phenomenon, termed entropic shielding, results in high protein repellant properties for surface-attached cross-linked hydrogels. The entropic shield arise as the swelling of the polymer network is anisotropic perpendicular to the surface. This anisotropic swelling leads to a high loss in conformational entropy (stretched polymer chains) and influences the entropy of mixing such that higher mixing would increase entropy but also decrease the conformational entropy, due to stronger stretching, making this situation energetically unfavorable. Therefore, when a protein is introduced to the layer, the further stretching of the polymer chains leads to a larger decrease in conformational entropy, making the penetration of the protein into the layer energetically unfavorable. Additionally, size exclusion prevents the penetration of networks with small mesh size by large macromolecules.

C,H insertion crosslinking (CHic) has been used to simultaneously form a hydrogel network and attach this network to a surface. Conventionally, a copolymer consisting of a hydrophilic monomer and a monomer containing crosslinker groups is deposited onto the surface to be coated. Through thermal or photochemical excitation, the reactive groups are transformed for example into ketyl biracdicals, nitrenes, or carbenes, which are able to react with nearby C,H thus forming a cross-linked network which becomes simultaneously attached to any organic surface.

Hydrogel precursor are conventionally applied to a surface by spin coating, dip coating, and doctor blading. Such coating methods are limited to the geometry of surfaces to which they can be applied. For example, such coating methods are not useful in effective coating the interiors containers with non-planer surfaces, such as tubes, chambers, and the like.

### SUMMARY

A process of forming a repellant surface attached hydrogel layer having reduced nonspecific adsorption of nontargeted species can include spray coating a hydrogel precursor solution onto a surface under conditions to generate a spray coated layer having a surface roughness with surface features with an average features height of less than 20 nm, wherein the hydrogel precursor solution comprises a hydrogel precursor dissolved in a solvent; drying the spray coated layer; and irradiating and/or heating the spray coated layer to crosslink the hydrogel precursor thereby forming the hydrogel and attaching the hydrogel to the surface.

A coated container having reduced nonspecific adsopriton of nonspecific targets can include an interior volume surrounded by one or more walls having one or more interior surfaces, and a coating comprising a spray coated hydrogel layer surface attached to the one or more interior surfaces. The spray coated hydrogel layer has a surface roughness with surface features having an average feature height of less than 20 nm.

### BRIEF DESCRIPTION OF THE DRAWINGS

FIG. 1 is a schematic illustration of a method of spray coating a surface attached hydrogel in accordance with the disclosure;

FIG. 2A is a graph showing spray coating layer thickness as a function of hydrogel precursor concentration in the sprayed hydrogel precursor solution;

FIG. 2B is a graph showing spray coating layer thickness as a function of feed pressure and vapor pressure at a constant hydrogel precursor concentration of 1 mg/ml;

FIGS. 3A to 3D are AFM topography images of spray coated PMMA slides showing roughness as a function of spraying parameters;

FIG. 4A is a fluorescent image of an untreated PMMA slide after incubation in fluorescent labeled goat anti-human IgA;

FIGS. 4B to 4D are fluorescent images of spray coated PMMA slides at different hydrogel precursor concentration after incubation in fluorescent labeled goat anti-human IgA;

FIG. 5 is a graph showing mean gray value as a function of feature height on spray coated PMMA slides showing the amount of fluorescent protein adsorbed on the surface;

FIG. 6 is a bar chart showing mean gray values for blood collection tubes spray coated with a hydrogel as compared to an uncoated blood collection tube;

FIG. 7 is a schematic illustration of HRP/TMB-based ELISA method for resting protein repellency;

FIG. 8 is a graph showing the reducing of nonspecific adsorption achieved with the coated surfaces of the disclosure as compared to uncoated surfaces; and

FIG. 9 is a bar chart of the integral of the absorbance peaks at 450 nm from a HRP/TMB ELISA for blood collection tubes pray coated in accordance with the disclosure with a solution of PDMMA-co-5% MABP in ethanol (20 mg/ml) and crosslinked versus uncoated blood collection tubes incubated for 1 hour with delipidized human serum (1:10 dilution).

### DETAILED DESCRIPTION

Spray coating is a technique by which a liquid material is dispersed into small drops. In general, the droplets are formed through the breakup of a liquid jet or sheet by disintegration through the liquid's own kinetic energy, through use of an external gas, or through mechanical means such as applying an external rotating or vibrating device. Disturbances induced in the liquid jet result in the formation of ligaments and eventually droplets. Through this process, a liquid film is deposited on the surface, which dries relatively rapidly leading to a broad spectrum of film topologies. Examples of physical processes leading to variations in the surface topology of a spray coated polymer film can include the coffee stain effect, the development of roughness features due to Bénard-Marangoni convection or vapor recoil, differential evaporation and solute capillarity. The surface roughness can be correlated with evaporation rates and polymer concentration. As a result of reduced mobility of a polymer system, patterns caused by these effects can be frozen in the dried polymer film.

It has advantageously been found that through the combination of spraying and thermal-or photo-activation, a spray deposited thin surface attached hydrogel layer can be formed on various geometries, such as the inside of a tube, vertically extending surfaces, and surfaces with curvature. This can be advantageous used for coating the inside of a blood collection tube, for example, which have vertically extending sidewalls and a narrow opening making it difficult to uniformly coat. It has been surprisingly found that the thickness and surface roughness of the spray coated hydrogel layer affects performance of the hydrogel layer in having reduced non-specific adsportion of non-targeted species, for example, having repellency to biological materials such as proteins. It has been found that surface roughness must be minimized through control of the spray coating parameters to achieve good repellant properties of the surface attached hydrogel layer. For example, the process can include spray coating with conditions to generate a spray coated layer having a surface roughness with surface features with an average feature height of less than 20 nm.

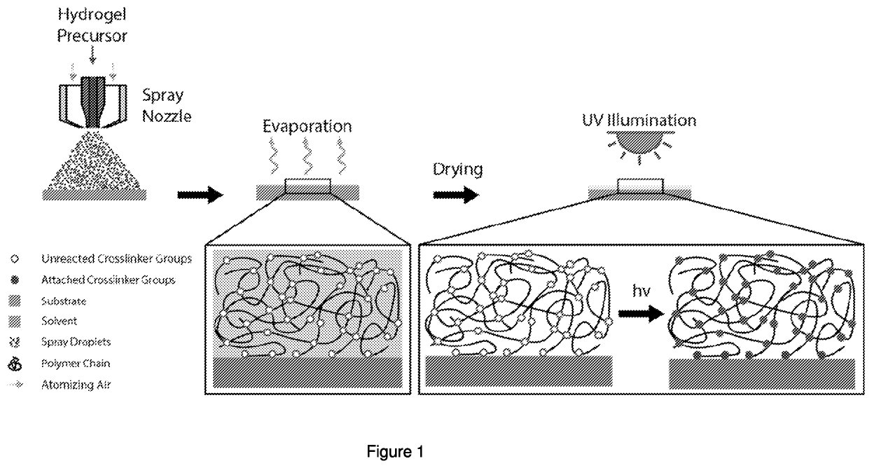

Referring to FIG. 1, a schematic example of forming a spray coated surface attached hydrogel layer in accordance with the disclosure is illustrated. The process can include applying through spray coating a liquid hydrogel precursor solution to coat a surface. The spray coated solution can be then be allowed to dry until the solvent from the hydrogel precursor solution evaporates. Once dried, the coating can be exposed to irradiation and/or heat to crosslink the hydrogel precursor and chemically attach the hydrogel to the surface. In embodiments such as shown in FIG. 1, the hydrogel precursor can be sprayed using an external mixing two fluid nozzle. Other spray drying systems or apparatuses can also be used.

Coated surfaces of the disclosure can include a repellant coating that includes a spray-coated hydrogel layer surface attached to the surface, with the spray coated hydrogel layer having a surface roughness with surface features having an average feature height of less than 20 nm. As used herein, “repellant” refers to a coating capable of preventing or reducing nonspecific adsorption of nontargeted species. For example, the repellant coating can prevent or reduce nonspecific adsorption of one or more biological materials. The surface to be coated can be, for example, an interior surface of a container having one or more walls that are oriented vertically and/or have a curvature. When applied to interior surfaces of containers, all or a portion of the interior walls or surfaces of the container can be coated.

Without intending to be bound by theory, it is believed that hydrogels having surface roughness with surface features having an average feature height greater than 20 nm may have reduced entropic shielding. Entropic shielding relies on high one-dimensional swelling of the hydrogel, which results in stretching of the polymer chains when a protein attempts to enter the layer, leading to a large drop in the entropy of conformation and creating an energetically unfavorable situation for proteins to enter the layer. High roughness surfaces could have swelling that occurs in the horizontal plane between the features. This reduces the stretching of the polymer chains away from rigid surfaces, which would lower the constriction of the polymer chains and reduce the drop in the entropy of confirmation. This is believed to lead to entropy of mixing being more dominant, resulting in non-targeted species, such as biological materials, entering the layer. The non-targeted species may be trapped in the layer, rather than physisorbed.

The hydrogel layer formed by the process of the disclosure can have, for example, a surface roughness with surface features having an average height of less than 20 nm, less than 15 nm, less than 10 nm, or less than 5 nm. Surface features have a height, which extend from an end adjacent to the surface which is coated to an oppositely disposed free end.

The liquid hydrogel precursor solution can include a hydrogel precursor that has at least two distinguishable monomer units—a hydrophilic (polar) but non-charged monomer unit (having no ionic groups) and a monomer unit that has at least one side group capable of crosslinking by C,H insertion crosslinking when exposed to irradiation or heat. For example, the hydrogel precursor can be a copolymer of poly(dimethylacrylamide and methacryloyloxybenzophenone). Suitable hydrophilic non-charged monomers can include acrylamides and acrylate derivatives. For example, suitable crosslinker monomers can include aromatic carbonyl groups, azides, diazirdines, diazocarbonyls, and diazocarboxyls. The precursor can include one or more of cross-linking monomers selected from methacryloyloxy benzophenone, 1-(2-(methacryloyloxy)ethyl) 3-methyl 2-diazomalonate, 2-(2-diazo-2-phenylacetoxy)ethyl methacrylate, and 2-acryloyloxythioxanthone.

The precursor can include from about 1 wt % to about 25 wt % or about 1 wt % to about 10 wt % of the cross-linking monomer unit, based on the total weight of the hydrogel precursor. For example, the hydrogel precursor can include a copolymer of poly(dimethylacryamide) and 5 wt % methacryloyloxybenzophenone. Other suitable amounts of cross-linker monomer unit include, by weight of the precursor, about 1, 2, 3, 4, 5, 6, 7, 8, 9, 10, 11, 12, 13, 14, 15, 16, 17, 18, 19, 20, 21, 22, 23, 24, and 25 wt % and any values there between and ranges defined between these values.

The precursor can include about 0.5 mol % to 10 mol %, or about 1 mol % to about 5 mol %, or about 4 mol % to about 8 mol % of the cross-linking repeat unit based on the total moles of hydrogel precursor. Other suitable mole percentages based on the total moles of the hydrogel precursor include about 0.5, 1, 1.5, 2, 2.5, 3, 3.5, 4, 4.5, 5, 5.5, 6, 6.5, 7, 7.5, 8, 8.5, 9, 9.5 and 10 mol %.

The hydrogel precursor can be included in the hydrogel precursor solution in an amount of about 0.5 mg/ml to about 100 mg/ml, about 1 mg/ml to about 10 mg/ml, about 20 mg/ml to about 75 mg/ml, about 50 mg/ml to about 100 mg/ml, or about 30 mg/ml to about 60 mg/ml. Other suitable concentrations include about 0.5, 0.6, 0.7, 0.8, 0.9, 1, 5, 10, 15, 20, 25, 30, 35, 40, 45, 50, 55, 60, 65, 70, 75, 80, 85, 90, 95, or 100 mg/ml and any values there between and ranges defined between these values.

The process can include adjustment of the spray coating time, feed pressure, and/or pressure of the vaporizing air to achieve the conditions suitable for providing a spray coated layer having a surface roughness with surface features having an average feature height of less than 20 nm. Adjustment of such parameters will depend on a given spray coating set up and selection of these parameters for a given setup to achieve the necessary spray coating conditions can be readily determined by the skilled person once informed of the target spray coating surface roughness.

The process can include spraying the hydrogel precursor solution for a spray time of about 0.1 second to about 1.5 second. The spray time can be selected to achieve a target thickness and can depend on the spray coating equipment set-up. For example, for a stationary set-up the spray time can be about 0.1 to 0. 5 seconds. Suitable spray times to achieve a spray coated layer having a desired thickness and/or surface roughness can be readily identified by a person skilled in the art for a given spray coating set-up.

The spray coating can be performed at a feed pressure of about 0.25 bar to about 1.5 bar. Other feed pressures can be contemplated herein for a given spray coating set-up and can be readily identified by the skilled person to achieve a desired thickness and/or surface roughness for that given spray coating setup.

The spray coating can be performed at a vapor pressure of about 0.5 bar to about 2 bar. The vapor pressure must be greater than the feed pressure. Other vapor pressures can be contemplated herein for a given spray coating set-up and can be readily identified by the skilled person to achieve a desired thickness and/or surface roughness for that given spray coating setup.

The surface attached hydrogel layers of the disclosure can advantageously prevent or reduce the amount of non-specific adsorption of a non-targeted species. For example, the surface attached hydrogel layers of the disclosure can prevent or reduce adsorption of biological materials, such as microorganisms, cells, and cell components. Cell components can include one or more of extracellular vesicles, cell debris, protein, protein complexes, glycoprotein, nucleic acid, and lipids. Cells can be nucleated cells, circulating tumor cells, thrombocytes, and erythrocytes.

The process and resulting hydrogel layers of the disclosure can reduce nonspecific adsorption by at least about 30%, at least about 40%, or at least about 50%, as compared to the surface having no hydrogel layer. For example, nonspecific adsorption can reduced by about 30% to about 90%, about 40% to about 60%, or about 50% to about 80%. Other percent reductions can include about 30%, 35%, 40%, 45%, 50%, 55%, 60%, 65%, 70%, 75%, 80%, 85% and 90% and values there between and ranges defined between these values.

The process can include spray coating the hydrogel precursor solution under conditions to achieve a spray coated layer having a thickness of about 10 nm to about 1000 nm, about 15 nm to about 50 nm, about 100 nm to about 1000 nm, about 75 nm to about 150 nm, about 600 nm to about 800 nm, or about 500 nm to about 900 nm. Other suitable thicknesses include about 10, 12, 14, 16, 18, 20, 22, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 nm and any values there between and ranges defined between these values.

The spray coated layer can be cross-linked by exposure to heat or irradiation. For example, the spray coated layer can be exposed to UV light for a period of time sufficient to cross-link the cross-linking monomers in the hydrogel precursor. The UV light can have a wavelength in a range of about 200 nm to about 400 nm, about 250 nm to about 400 nm, about 254 to about 400 nm, about 250 nm to about 260 nm, or about 360 nm to about 370 nm. Other suitable wavelengths can include about 200, 205, 215, 220, 225, 230, 235, 240, 245, 250, 254, 255, 260, 265, 270, 275, 280, 285, 290, 295, 300, 305, 310, 315, 320, 325, 330, 335, 340, 345, 350, 355, 360, 370, 375, 380, 385, 390, and 400 nm and any values there between and ranges defined between these values. For example, the hydrogel precursor can be adapted to cross-link using a common UV light source, which has an emission line at 254 nm and an emission line at 365 nm. Crosslinking can be additionally or alternatively achieved through heating of the spray coated layer. The spray coated layer can be heated, for example, to a temperature of about 60° C. to about 200° C., about 60° C. to about 120° C., about 75° C. to about 90° C., about 100° C. to about 200° C., or about 125° C. to about 175° C. Other suitable temperatures include, for example, about 60, 62, 64, 66, 68, 70, 72, 74, 76, 78, 80, 82, 84, 86, 88, 90 92, 94, 96, 98 100, 105, 110, 115, 120, 125, 130, 135, 140, 145, 150, 155, 160, 165, 170, 175, 180, 185, 190, 195, and 200, and any values there between and ranges defined by these values.

A variety of surfaces can be treated with the hydrogel to achieve a reduction in the non-specific adsorption of a non-targeted species. For example, the surface to be coated can be glass or polymeric. The polymeric surface can be for example a plastic. The surface can be formed of or composed of, for example, PET.

Processes of the disclosure can be particularly advantageous for achieving coatings on curved surfaces and vertically extending surfaces, but application of the process to planar surfaces is also contemplated herein. For example, the surface can be an internal surface of a tube, container, jar, or other package. Such internal surface can include planar surfaces, such as vertical side walls or a horizontal bottom surface, but also such as a curved bottom wall or curvature of the side walls. For example, the surface can be an internal surface of a biological sample collection tube. Processes of the disclosure can be used to coat interior surfaces for a variety of containers and is not limited to any particular container. Examples of containers can include tubes, vacutainers, vacuettes, beakers, cups, and the like.

Processes of the disclosure can be advantageous where the surface to be coated is an internal surface and the opening through which coating is to be achieved is relatively narrower. For example, the surface to be coated can be internal surface of a container having an opening of about 4 mm to about 75 mm, or about 4.8 mm to about 72 mm.

For some surfaces, such as glass surfaces, the process can further include pretreating the surface prior to spray coating with the hydrogel precursor solution. Any surface treatment that results in covalently bound C,H groups on the surface can be used. The C,H groups are useful for the crosslinking by C,H insertion. The pretreatment can include for example, treating the surface with triethoxysilane benzophenone to form a layer on the surface, for example a self-assembled monolayer. The pretreatment can include forming a silane layer on the surface. The silane layer can be for example, but not limited, to benzophenone silane, which provides for active binding when cross-linking the hydrogel. Alternatively, the silane layer can be for example, but not limited to, alkylsilane, which provides for passive binding during crosslinking of the hydrogel. For some surface, such as surface made from polymers, the C,H groups needed for the C,H insertion are already present on the surface and pretreatment is not needed. Examples of pretreatments can include, but are not limited, trichlorosilane, triethoxysilane, hexamethyldisilazane, and triethoxysilane benzophenone.

It has been found that processes of the disclosure can allow for coating of interior surfaces which cannot otherwise be effectively or efficiently coated using other methods. For example, traditional dip coating cannot be used to coat an interior surface of a tube or other interior surface with vertical walls and/or curvatures as a uniform coating cannot be effectively achieved. Other techniques such as dip coating or modified dip coating methods may be useful in achieving more uniform coatings, but requires filling of the entire container and removal of the excess, which result in significant waste of the coating material leading to increased cost. Processes of the disclosure can quickly and effectively coat interiors of surfaces, such as tubes, with less initial precursor material required as compared to traditional methods, significant reduction in the amount of waste of coating material, and while achieving highly uniform coatings with effective reduction of nonspecific adsorption.

### EXAMPLES

All chemicals were obtained from Sigma-Aldrich or Carl Roth and used as received. Alexa Fluor 647-conjugated AffiniPure goat anti-human serum IgA was obtained from Jackson ImmunoResearch laboratories, Inc.

Dip coating in the comparative examples was achieved using a Z 2.5 tensile test machine from Zwick GmbH.

Spray coating was done using a Krautzberger Mikro 3 spray gun with an external mixing two-fluid nozzle. Spraying of blood collection tubes was completed using this spray gun with a custom 200 mm rotary nozzle from Krautzberger. The length of the tube was coated by lowering and raising the tube while spraying using an EPCO-16-200-8P-ST-EB-KF+EAGF-P1-KF-16-200 motor with a CMMO-ST-C5-1-D108 controller from Festo.

UV crosslinking of flat substrates was done using a Stratagene Stratalinker 2400. UV crosslinking of blood collection tubes was done using a Bio-Link 365 UV crosslinker with a mirrored interior from Vilber.

A SE 400adv ellipsometer from Sentech Instruments was used for ellipsometry measurements. AFM measurements were done with a Nanowizard 4 AFM from JPK Instruments AG with Si ACL-W cantilevers (resonance frequency of 145-230 Hz and a force constant of 20-95 N/m) from AppNano. Fluorescent imaging was done with a SensoSpot® Fluorescence Microarray Reader from Sensovation.

### Example 1: Spray Coating Hydrogel on PMMA slides

The hydrogel layers were prepared on PMMA slides by spray coating. A hydrogel precursor solution was prepared by dissolving a copolymer of poly(dimethylacrylate) (PDMAA) and 5% methacryloyloxybenzophenone (MABP) in ethanol at various concentrations. The slides were lain horizontally in a convection-controlled spray box with the spray gun positioned 10 cm above, facing down. An external mixing two-fluid nozzle was used to spray the hydrogel precursor solution. The nozzle used atomizing air to cause disturbances on the liquid jet of the hydrogel precursor solution, leading to a breakup of the jet and the formation of droplets. The process was controlled by adjusting the atomizing air pressure (vapor pressure). A pressure was also applied on the spray, which is the feed pressure. This allowed adjustments of the mass flow rate of the spray. The spraying time was 200 ms. Hydrogen precursor concentration, vapor pressure, and feed pressure were varied as described in detail below.

After spraying the samples were allowed to dry in the spray box with the fume hood open. After drying, the samples were illuminated with 1 J/cm² of UV-light at 254 nm to form the hydrogel network and attach it to the surface. Following crosslinking, the slides were placed in a cuvette filled with ethanol on a shaker table for 5 minutes and then dried.

Surface Topography: Surface topography measurements of the spray coated samples were done on the coated PMMA slides by taking 20×20 μm scans in air using AFM in AC tapping mode. Image processing was completed using Gwyddion AFM software. Values for the size of surface features on the spray coated layers were obtained by calculating the average of the height of the scan area (Average Value in Gwyddion), indicating the average difference in height between valleys and peaks on surface. Spray coated samples are generally rougher than dip coated samples, which are extremely smooth and have an average feature size of 1 nm or less.

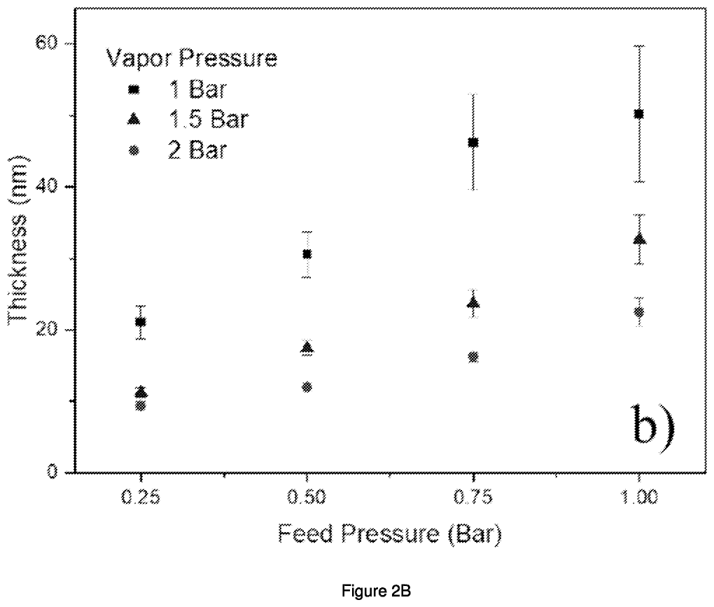

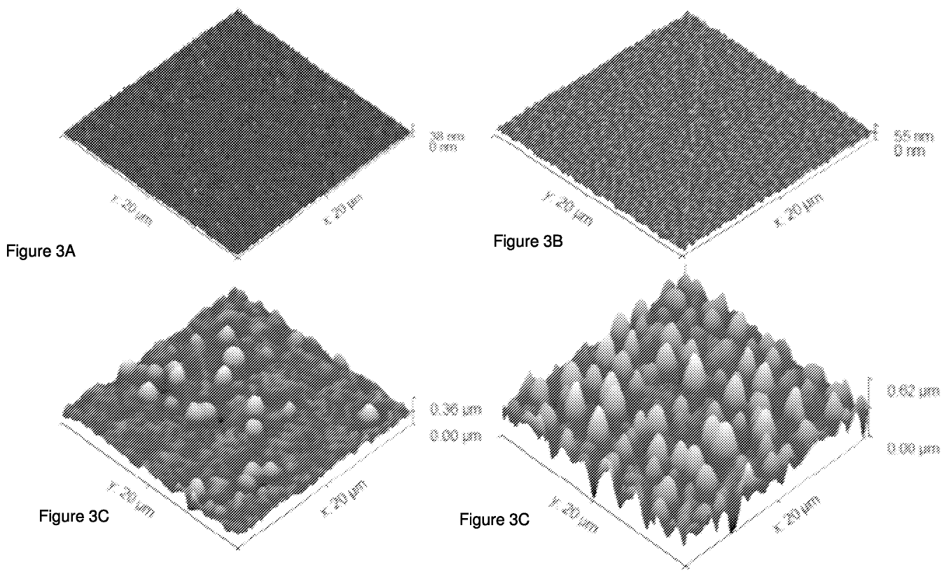

FIGS. 3A to 3D show the degree of roughness of the final spray coated hydrogel surface was as a function of concentration. Each of the samples shown in FIGS. 3A to 3D were sprayed using a feed pressure of 0.5, vapor pressure of 1 bar, and spray time of 0.2 seconds. The sample of FIG. 3A had a hydrogel precursor solution with a concentration of 1 mg/ml. The sample of FIG. 3B had a hydrogel precursor solution with a concentration of 5 mg/ml. The sample of FIG. 3C had a hydrogel precursor solution with a concentration of 12.5 mg/ml. The sample of FIG. 3D had a hydrogel precursor solution with a concentration of 20 mg/ml.

An increase in the size of the features was found corresponding to an increase in the polymer concentration. The rms roughness (Sq) ranged from 2nm for the 1 mg/ml sample in FIG. 3A to 80 nm for the 20 mg/ml sample in FIG. 3D, with a roughly linear increase with concentrations. The samples generated from low concentration solutions (1 mg/ml and 5 mg/ml) did not show distinct surface features (FIGS. 3A and 3B). The samples generated from hydrogel precursor solutions having a concentration of 20 mg/ml had average features sizes of about 0.5 μm. Given the size of the features, it is unlikely they reflect the initial droplet size. The droplets of hydrogel precursor solution produced by the spray apparatus used in this example were expected to range between 10-200 μm. Droplet size was not expected to vary much with concentration of the hydrogel precursor solution. Without intending to be bound by theory, it is believed that the features formed during the drying of the liquid film deposited on the surface after spraying. The amount of roughness that formed had a correlation with the amount of material deposited on the surface during spraying, with rougher layers having the highest thicknesses. While increase thickness of the drying a spray solution, as a result of depositing more material, has been found to influence the production of surface patterns, observations have not been previously made regarding effect on surface roughness. It has been beneficially found that the concentration can affect the resulting surface roughness.

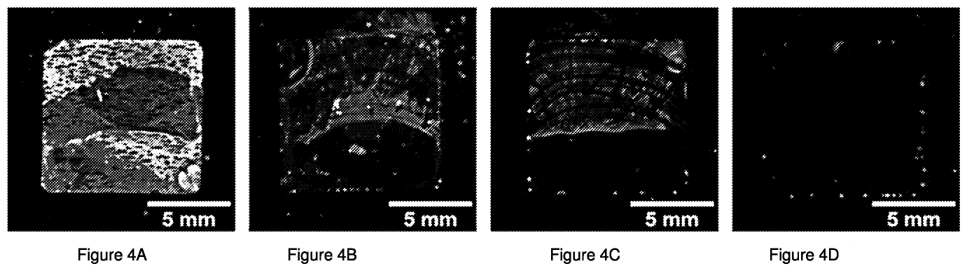

Protein Adsorption: Protein adsorption for the coated PMMA slides was done by incubating the hydrogel layers with Alexa Fluor 647-conjugated AfiniPure goat anti-human Serum IgA at a concentration of 1 μg/ml in phosphate-buffered saline (PBS). A 1×1 cm frame seal was applied to the hydrogel layer, into which 25 μl of protein solution was pipetted. The frame seal covered was applied such that no air bubbles were visible. The samples were incubated for 1 hour. After incubation, the samples were washed with PBS-0.1% Tween 20 and PBS for 5 minutes each on a shaker table. Following washing, the samples were dried using compressed air. Fluorescent images were taken using a microarray reader with the red channel and an exposure time of 400 ms. FIG. 4 illustrates the protein adsorption tests on three samples having varying concentrations of the hydrogel precursor in the hydrogel precursor solution. FIG. 4A shows an untreated sample. FIG. 4B shows a sample spray coated with a hydrogel precursor solution having a concentration of 10 mg/ml. FIG. 4C shows a sample spray coated with a hydrogel precursor solution having a concentration of 5 mg/ml. FIG. 4D shows a sample spray coated with a hydrogel precursor solution having a concentration of 1 mg/ml. In each spray coated sample a feed pressure of 0.5 bar, a vapor pressure of 1 bar, and a spray time of 0.2 seconds was used.

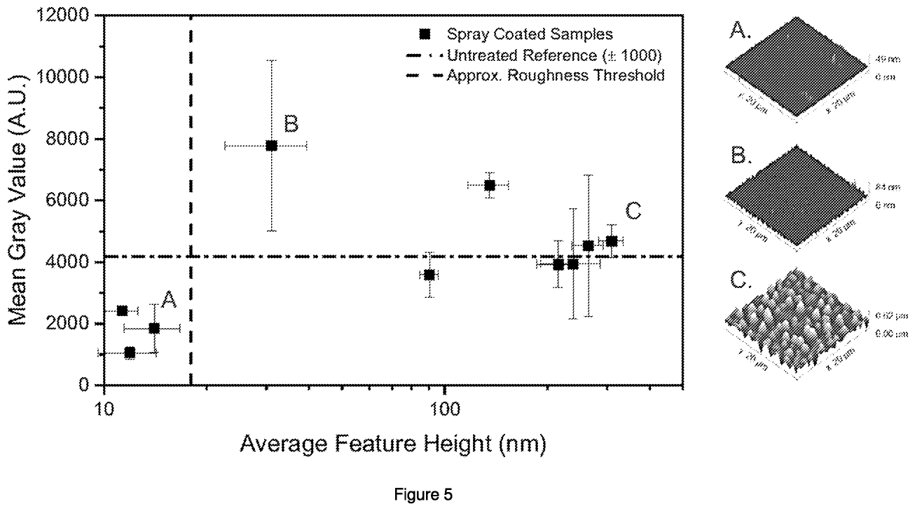

It was surprisingly found that surface topography of the spay coated film affected the protein repellency. As illustrated in FIG. 5, smooth hydrogel layers showed good protein repellency with significant improvement over the uncoated control sample. In samples having rougher layers (samples b and c in FIG. 5), which had features with a feature size above about 15-20 nm, the protein repellent function of the spray coating completely disappeared. Without intending to be bound by theory, it is believed that the observed correlation that protein repellency diminishes as a function of increased surface roughness may be due to the polymer swelling in the roughness features in a three-dimensional way, whereas in a continuous smooth layer, there is two-dimensional swelling. The three-dimensional swelling leads to stretching of the polymer chains in the hydrogel and lower entropic shielding.

### Comparative Example 1: Dip Coating Hydrogel on PMMA Slides

The hydrogel layers were prepared on PMMA slides by dip coating. As in Example 1, a copolymer of poly(dimethylacrylate) (PDMAA) and 5% methacryloyloxybenzophenone (MABP) was dissolved in ethanol. Dip coating was done at a draw speed of 100 mm/min and a polymer concentration of 10 mg/ml. The dip-coated slides were allowed to dry in ambient conditions and then cross-linked using 1 J/cm² of UV-light at 254 nm as described in Example 1.

Protein Adsorption: Protein adsorption was tested on the dip coated samples using the procedure outline in Example 1.

### Example 2: Spray Coating Hydrogel in Blood Collection Tubes

Glass blood collection tubes were primed with a self-assembled monolayer to provide C,H groups for the CHic reaction. For this, triethyoxysilane benzophenone was synthesized and dissolved in toluene with a concentration of 10 mg/ml. 5 ml of this solution was added to the blood collection tube and coated long the walls using a vortex mixer. The 5 ml was then poured into subsequent tubes to be coated with the SAM and dispersed on the walls of the tube using a vortex mixer. The tubes were placed upside down in an oven for 30 minutes at a temperature of 120° C. Following the heat treatment, the tubes were rinsed with toluene and dried upside down.

The hydrogel precursor solution was prepared by dissolving a copolymer of poly(dimethylacrylamide (PDMMA) and 5% methacryloyloxybenzophenone (MABP) in ethanol at a concentration of 20 mg/ml. The tubes were placed in a holder and sprayed along the length of the tube. The vapor and feed pressures used were 1.4 Bar and 0.7 Bar, respectively. The tubes were dried upside down in ambient air for about 30 minutes.

After drying, the tubers were illuminated with 10 J/cm³ of UV-light at 365 nm to form the hydrogel network and to attach the hydrogel to the surface. Following crosslinking, the tubes were washed with ethanol and dried in a centrifuge.

Protein Adsorption: Protein adsorption was tested by adding 5 ml of 1 mg/ml solution of bovine serum albumin (BSA), Cy5 in PBS buffer to coated and uncoated tubes. The tubes were incubated for 1 hour, after which the protein solution was poured out and the tubes were washed 3 times with PBS-0.1% Tween 20 and 3 times with PBS. After washing, the tubes were dried using a centrifuge. The adsorption of protein was measured by taking fluorescent images using a microarray reader with the red channel and an exposure time of 200 ms. Images for flat substrates and BCTs were processed and mean gray values were obtained using ImageJ software.

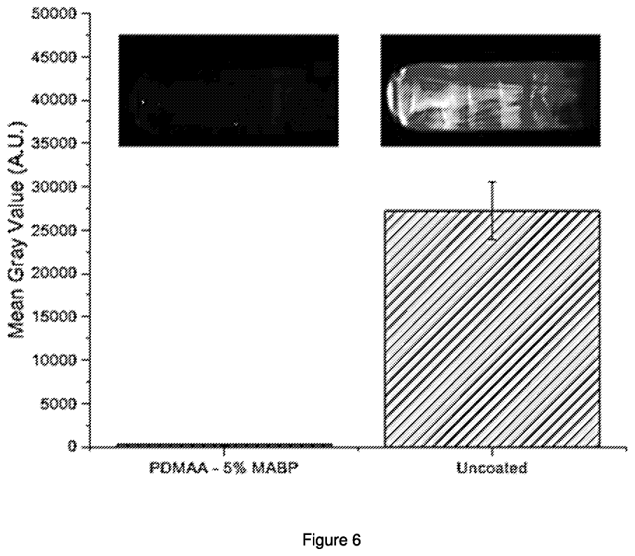

FIG. 6 shows the fluorescent images and corresponding mean gray values for the spray coated blood collection tube as compared to an uncoated due. It was found that the coated tube resulted in a surface with low adsorption of BSA compared to the uncoated tube with over 10× the adsorption.

### Protein Adsorption HRP/TMB-Based ELISA Method

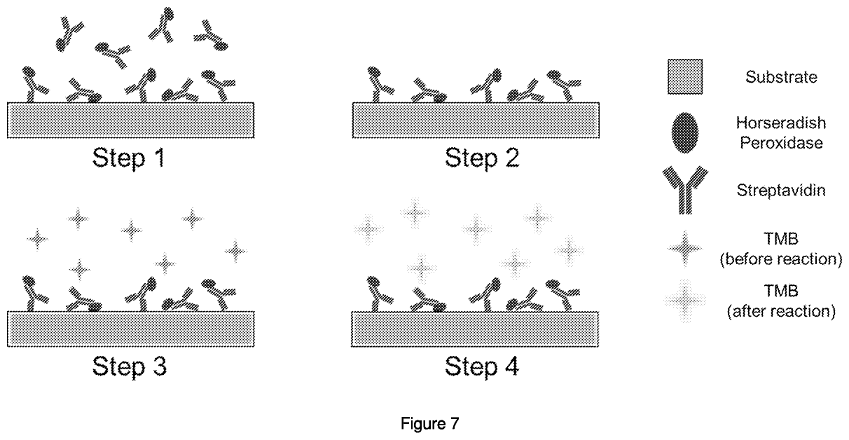

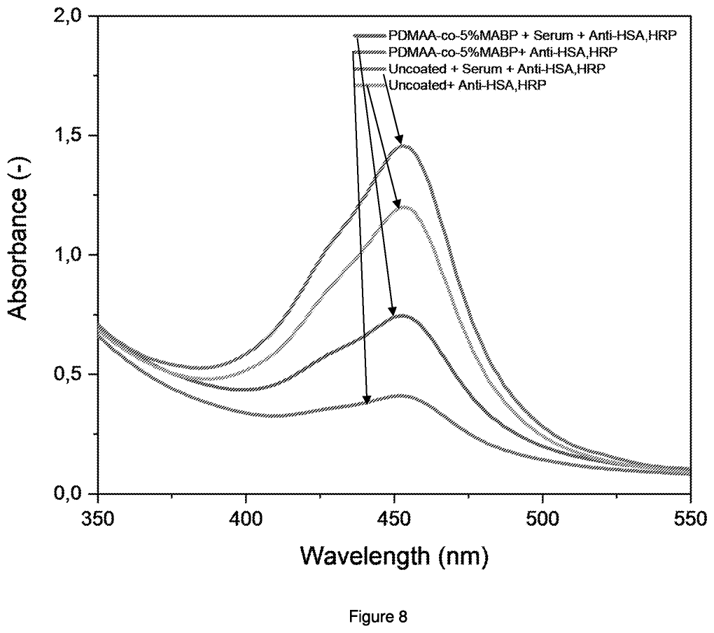

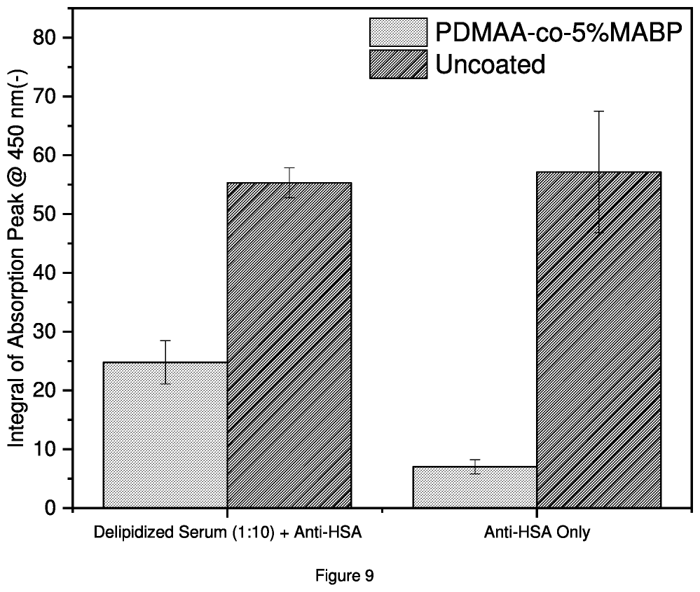

The coated blood collection tubes were also tested for protein repellency using horseradish peroxidase (HRP) and 3,3′,5,5′-Tetramethylbenzidine (TMB) interaction to detect the presence of adsorbed proteins using UV-Vis. FIG. 7 is a schematic illustration of the test method. Coated and uncoated blood collection tubes were incubated in delipidized serum (1:10 dilution) for 1 hour. After incubation, the tubes were washed three times with PBS-Tween 20 and then once with PBS. Human Serum Albumin antibodies conjugated with HRP were added to the tubes and incubated for 2 hours followed by washing. TMB was then added to the tubes, which reacts with HRP immobilized on the surface. H₂SO₄ stopped the reaction. Blood collection tubes in accordance with the disclosure were spay coated with a solution of PDMAA-co-5% MABP in ethanol (20 mg/ml) using a feed pressure of 0.7 bar and a vapor pressure of 1.4 bar. Absorbance was measured using UV-Vis (350-550 nm range). A high signal at 450 nm indicated more HRP, which is indicative of more protein adsorption. As illustrated in FIG. 8, the coated samples exhibited a lower signals at 450 nm indicating that the coating resulted in reduced protein adsorption. FIG. 9 illustrates that a 50% reduction in absorbance was observed for the coated blood collection tubes incubated in human serum as compared to the uncoated samples.

### Example 3: Thickness Measurements

The thickness of the spray coating was measured by spray coating silicon wafers with the hydrogel precursor solution as described in Example 1, omitting the crosslinking and washing steps. The silicon slides were polished on one side and cut to 1.5×2 cm rectangles. Film thickness was measured using ellipsometry.

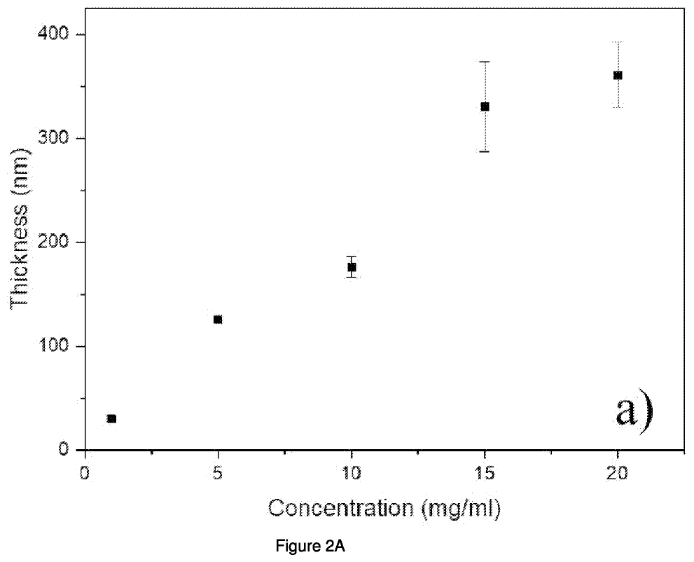

FIG. 2A shows the effect of the concentration of the hydrogel precursor solution on the layer thickness. The samples shown in FIG. 2A were all sprayed using a feed pressure of 0.5 bar and a vapor pressure of 1 bar. Spray time was 0.2 seconds. A roughly linear trend was seen, with the average thickness ranging between 30 and 400 nm. Increasing concentrations corresponded to more sprayed polymer resulted in thicker deposited layers.

FIG. 2B illustrates the variation in thickness resulting from varying the feed pressure while using a hydrogel precursor solution having a concentration of 1 mg/ml. Spray time was 0.2 seconds. A linear increase in thickness was observed as the feed pressure was increased. FIG. 2B also illustrates the effect of varying vapor pressure. In contrast to feed pressure, increasing vapor pressure was found to result in reduction in thickness. Without intending to be bound by theory, it is believed that the reduction is due to the production of smaller droplets at higher vapor pressures which may be carried away by the sheet gas and do not reach the surface, and/or due to increased backpressure resulting in less material being sprayed.

It has been found that films with controlled thicknesses ranging from 10 to 400 nm can be obtained through control of one or more of hydrogel precursor concentration, feed pressure, and vapor pressure.

### REFERENCES

1. Scherag, F. D.; Niestroj-Pahl, R.; Krusekopf, S.; Lücke, K.; Brandstetter. T.; Rühe, J. Highly Selective Capture Surfaces on Medical Wires for Fishing Tumor Cells in Whole Blood. Anal. Chem. 2017, 89 (3). 1846-1854.
2. Wörz, A.; Berchtold, B.; Moosmann, K.; Prucker, O.; Rühe, J. Protein-Resistant Polymer Surfaces. J. Mater. Chem. 2012, 22 (37), 19547.
3. Prucker, O.; Brandstetter, T.; Rühe, J. Surface-Attached Hydrogel Coarings via C,H-Insertion Crosslinking for Biomedical and Bioanalytical Applications (Review). Biointerphases 2018, 13 (1), 010801.
4. Moschallski, M.; Evers, A.; Brandstetter, T.; Rühe, J. Sensitivity of Microarray Based Immunoassays Using Surface-Attached Hydrogels. Anal. Chim. Acta 2013, 781, 72-79.
5. Park, K.; Mosher, D. F.; Cooper, S. L. Acute Surface-induced Thrombosis in the Canine Ex Vivo Model: Importance of Protein Composition of the Initial Monolayer and Platelet Activation. J. Biomed. Mater. Res. 1986, 20 (5), 589-612.
6. Rühe, J.; Biesalski, M. Biocompatibility of Microsystems. In Comprehensive Microsystems; Gíauchandani, Y., Tabata, O., Zappe, H., Eds.; Elsevier, 2008; pp 107-130.
7. Jeon, S. L.; Lee, J. H.; Andrade, J. D.; De Gennes, P. G. Protein-Surface Interactions in the Presence of Polyethylene Oxide. I. Simplified Theory. J. Colloid Interface Sci. 1991, 142 (1), 149-158.
8. Szleifer, I. Protein Adsorption on Surfaces with Grafted Polymers: A Theoretical Approach. Biophys. J. 1997, 72 (2 1). 595-612.
9. Halperin, A.; Fragneto, O.; Schollier, A.; Sferrazza, M. Primary versus Ternary Adsorption of Proteins onto PEG Brushes. Langmuir 2007, 23 (21), 10603-10617.
10. Pandiyarajan, C. K.; Prucker, O.; Zieger, B.; Rühe, J. Influence of the Molecular Structure of Surface-Attached Poly(N-Alkyl Acrylamide) Coatings on the Interaction of Surfaces with Proteins, Cells and Blood Platelets. Macromol. Biosci. 2013, 13 (7). 873-884.
11. Baghai, M.; Tamura, N.; Beyersdorf, F.; Henze, M.; Prucker, O.; Rühe, J.; Goto, S.; Zieger, B.; Heilmann, C. Platelet Repellent Properties of Hydrogel Coatings on Polyurethane-Coated Glass Surfaces. ASAIO J. 2014, 60 (5), 587-593.
12. Pérez-Perrino, M.; Navarro, R.; Prucker, O.; Rüthe, J. Binding of Functionalized Polymers to Surface-Attached Polymer Networks Containing Reactive Groups. Macromolecules 2014, 47 (8), 2695-2702.
13. Schuh, K.; Prucker, O.; Rühe, J. Surface Attached Polymer Networks through Thermally Induced Cross-Linking of Sulfonyl Azide Group Containing Polymers. Macromolecules 2008. 41 (23), 9284-9289.
14. Schenderlein, H.; Voss, A.; Stark, R. W.; Biesalski, M. Preparation and Characterization of Light-Switchable Polymer Networks Attached to Solid Substrates. Langmuir 2013, 29 (14), 4525-4534.
15. Toomey, R.; Freidank, D.; Rühe, J. Swelling Behavior of Thin, Surface-Attached Polymer Networks. Macromolecules 2004. 37 (3), 882-887
16. Schuh, K.; Prucker, O.; Rühe, J. Tailor-Made Polymer Multilayers. Adv. Funct. Mater. 2013, 23 (48), 6019-6023.
17. Körner, M.; Prucker, O.; Rahe, J. Kinetics of the Generation of Surface-Attached Polymer Networks through C, H-Insertion Reactions. Macromolecules 2016. 49 (7), 2438-2447.
18. Norrman, K.; Ghanbari-Siahkali, A.; Larsen, N. B. Studies of Spin-Coated Polymer Films. Annual Reports on the Progress of Chemistry—Section C. The Royal Society of Chemistry October 12, 2005. pp 174-201.
19. Krebs, F. C. Fabrication and Processing of Polymer Solar Cells: A Review of Printing and Coating Techniques. Solar Energy Materials and Solar Cells. Elsevier April 1, 2009, pp 394-412.
20. Brinker, C. J.; Frye. G. C.; Hurd, A. J.; Ashley, C. S. Fundamentals of Sol-Gel Dip Coating, Thin Solid Films 1991, 201 (1), 97-108.
21. Lefebvre, A.; Mcdonell, V. Atomization and Sprays. Second Edition.: 2017.
22. Handbook of Atomization and Sprays: Theory and Applications; Ashgriz, N., Ed.; Springer Science & Business Media, 2011.
23. Girotto, C.; Rand, B. P.; Genoe, J.; Heremans, P. Exploring Spray Coating as a Deposition Technique for the Fabrication of Solution-Processed Solar Cells. Sol. Energy Mater: Sol. Cells 2009. 93 (4), 454-458.
24. Rayleigh, Lord. On the Instability of Jets. Proc. London Math. Soc. 1878, s1-10 (1), 4-13.
25. Deegan, R. D.; Bakajin, O.; Dupont, T. F.; Huber, G.; Nagel, S. R.; Witten, T. A. Capillary Flow as the Cause of Ring Stains from Dried Liquid Drops. Nature 1997, 389 (6653), 827-829.
26. Deegan, R. D.; Bakajin, O.; Dupont, T. F.; Huber, G.; Nagel, S. R.; Witten, T. A. Contact Line Deposits in an Evaporating Drop. Phys. Rev. E—Stat. Physics, Plasmas, Fluids, Relat. Interdiscip. Top. 2000, 62 (1 B). 756-765.
27. Grigoriev, R. O. Control of Evaporatively Driven Instabilities of Thin Liquid Films. Phys. Fluids 2002, 14 (6), 1895-1909.
28. De Gennes, P. G. Instabilities during the Evaporation of a Film: Non-Glassy Polymer+Volatile Solvent. Eur. Phys. J. E 2001, 6 (5), 421-424.
29. Pearson, J. R. A. On Convection Cells Induced by Surface Tension. J. Fluid Mech. 1958, 4 (5), 489-500.
30. Probstein, R. F. Physicochemical Hydrodynamics: An Introduction; Elsevier Science, 1973.
31. Strawhecker, K. E.; Kumar, S. K.; Douglas, J. F.; Karim, A. The Critical Role of Solvent Evaporation on the Roughness of Spin-Cast Polymer Films [1]. Macromolecules 2001, 34 (14). 4669-4672.
32. Bassou, N.; Rharbi, Y. Role of Bénard-Marangoni Instabilities during Solvent Evaporation in Polymer Surface Corrugations, Langmuir 2009, 25 (1), 624-632.
33. Li, G.; Yao, Y.: Yang, H.; Shrotriya, V.; Yang, G.; Yang, Y. “Solvent Annealing” Effect in Polymer Solar Cells Based on Poly(3-Hexylthiophene) and Methanofullerenes. Adv. Funct. Mater. 2007, 17 (10), 1636-1644.
34. Bormashenko, E.; Pogreb, R.; Musin, A.; Stanevsky, O.; Bormashenko, Y.: Whyman, G.; Gendelman, O.; Barkay, Z. Self-Assembly in Evaporated Polymer Solutions: Influence of the Solution Concentration, J. Colloid Interface Sci. 2006, 297 (2), 534-540.
35. Mitov, Z.; Kumacheva, E. Convection-Induced Patterns in Phase-Separating Polymeric Fluids. Phys. Rev. Lett. 1998, 81 (16), 3427-3430.
36. Prucker, O.; Naumann, C. A.; Rühe, J.; Knoll, W.; Frank, C. W. Photochemical Attachment of Polymer Films to Solid Surfaces via Monolayers of Benzophenone Derivatives. J. Am. Chem. Soc. 1999, 121 (38), 8766-8770.
37. Kooij, S.; Sijs, R.; Denn, M. M.; Villermaux, E.; Bonn, D. What Determines the Drop Size in Sprays? Phys. Rev. X 2018, 8 (3).

What is claimed:

1. A process of forming a repellant surface attached hydrogel layer having reduced nonspecific adsorption of nontargeted species, comprising:
spray coating a hydrogel precursor solution onto a surface under conditions to generate a spray coated layer having a surface roughness with surface features with an average features height of less than 20 nm, wherein the hydrogel precursor solution comprises a hydrogel precursor dissolved in a solvent;
drying the spray coated layer; and
irradiating and/or heating the spray coated layer to crosslink the hydrogel precursor thereby forming the hydrogel and attaching the hydrogel to the surface.

2. The process of claim 1, comprising pretreating the surface with triethyoxysilane benzophenone, trichlorosilane, triethoxysilane, hexamethyldisilazane to form a layer prior to spray coating the hydrogel precursor solution.

3. The process of claim 1 or 2, wherein the hydrogel precursor comprises at least first and second monomer units, the first monomer unit being hydrophilic and non-charged and the second monomer unit having at least one side group capable of crosslinking.

4. The process of claim 3, wherein the first monomer unit is an acrylamide or acrylate derivative.

5. The process of claim 3 or 4, wherein the second monomer unit comprises at least one side group capable of cross-linking through C,H insertion.

6. The process of claim 5, wherein the second monomer unit is one or more of methacryloyloxy benzophenone, 1-(2-(methyacryloyloxy)ethyl) 3-methyl 2-diazomalonate, 2-(2-diazo-2-phenylacetoxy)ethyl methacrylate, and 2-acryloyloxythioxanthone.

7. The process of any one of the preceding claims, wherein the hydrogel precursor solution comprises a copolymer of poly(dimethylacrylamide) and 5% methacryloyloxybenzophenone dissolved in ethanol.

8. The process of any one of the preceding claims, wherein the hydrogel precursor is present in the hydrogel precursor solution at a concentration of about 0.5 mg/ml to about 100 mg/ml.

9. The process of claim 8, wherein the hydrogel precursor concentration is about 1 mg/ml.

10. The process of any one of the preceding claims, wherein the dried spray coated layer has a thickness of about 10 nm to about 1000 nm.

11. The process of any one of the preceding claims, wherein the surface attached hydrogel layer results in an at least 30% reduction in nonspecific adsorption of nontargeted species as compared to an uncoated surface.

12. The process of anyone of the preceding claims, comprising irradiating the spray coated layer comprises exposing the spray coated layer to UV light to crosslink the hydrogel precursor.

13. The process of claim 12, wherein the UV light has a wavelength in a range of about 200 nm to about 400 nm.

14. The process of claim 13, wherein the UV light has a wavelength of about 254 nm and/or 365 nm.

15. The process of any one of the preceding claims, wherein the surface has a curvature.

16. The process of any one of the preceding claims, wherein the surface is an internal surface of a container.

17. The process of claim 16, wherein the container is a tube, jar, or cup.

18. The process of claim 17, wherein the container is a blood collection tube.

19. The process of any one of the preceding claims, comprising heating the spray coated layer to crosslink the hydrogel precursor.

20. The process of claim 19, comprises heating the spray coated layer to a temperature of about 60° C. to 200° C.

21. The process of any one of the preceding claims, comprising heating and irradiating the spray coated layer to crosslink the hydrogel precursor.

22. The process of any one of the preceding claims, wherein irradiating or heating the spray coated layer crosslinks the hydrogel precursor and attaches the hydrogel to the surface by C,H insertion crosslinking.

23. The process of any one of the preceding claims, wherein the surface is glass.

24. The process of any one of the preceding claims, wherein the surface comprises a polymer.

25. The process of claim 24, wherein the surface is PET.

26. The process of any one of the preceding claims, comprising spraying coating the hydrogel precursor solution for a spray time of about 0.1 seconds to about 1.5 seconds.

27. The process of any one of the preceding claims, comprising spray coating the hydrogel precursor solution onto the surface under conditions to generate the spray coated layer to have surface features with an averages features size of less than 15 nm.

28. The process of any one of the preceding claims, comprising spray coating the hydrogel precursor solution at a feed pressure of about 0.25 bar to about 1.5 bar.

29. The process of any one of the preceding claims, comprising spray coating the hydrogel precursor solution at a vapor pressure of about 0.5 bar to about 2 bar.

30. The process of any one of the preceding claims, wherein the nontargeted species comprises biological material.

31. The process of claim 30, wherein the biological material comprises one or more of microorganisms, cells, and cell components.

32. The process of claim 31, wherein the cell components comprise one or more of extracellular vesicle, cell debris, protein, protein complexes, glycoprotein, nucleic acid, and lipids.

33. The process of claim 31 or 32, wherein the biological material comprises one or more of nucleated cells, circulating tumor cells, thrombocytes, and erythrocytes.

34. A coated container having reduced nonspecific adsorption of nonspecific targets, comprising:
an interior volume surrounded by one or more walls having one or more interior surfaces, and
a coating comprising a spray-coated hydrogel layer surface attached to the one or more interior surfaces, the spray-coated hydrogel having surface roughness with surface features with an average features height of less than 20 nm.

35. The container of claim 34, wherein the container has an opening of about 4 mm to about 75 mm through which the interior surface is accessed.

36. The container of claim 34 or 35, wherein the container is a blood collection tube.

37. The container of any one of claims 34 to 36, wherein the hydrogel layer has a thickness of about 10 nm to about 1000 nm.

38. The container of any one of claims 34 to 37, wherein the coating results in an at least 30% reduction in nonspecific adsorption of nontargeted species as compared to an uncoated surface.

39. The container of claim 38, wherein the nontargeted species comprises biological material.

40. The container of claim 39, wherein the biological material comprises one or more of microorganisms, cells, and cell components.

41. The container of claim 40, wherein the cell components comprise one or more of extracellular vesicle, cell debris, protein, protein complexes, glycoprotein, nucleic acid, and lipids.

42. The container of claim 40 or 41, wherein the biological material comprises nucleated cells, circulating tumor cells, thrombocytes, and erythrocytes.

43. The container of any one of claims 34 to 42, wherein the one or more interior surfaces extend in at least two planes.

44. The container of any one of claims 34 to 43, wherein at least one of the one or more interior surfaces has a curvature.

45. The container of any one of claims 34 to 44, wherein the container is a blood collection tube and the hydrogel is coated on at least interior surfaces of the side wall of the blood collection tube, and the hydrogel layer has a substantially uniform thickness on the interior surfaces of the side wall of the blood collection tube.
